SEQUENCE LISTING (3 sequences)

SEQ ID NO: 1 - DNA, 1932 bp, mouse
  CDS (52)..(1890)
tgcgtggaaggaaaaggaaggagcgggagcttccgaggagtgatcgccgaaatgttccggtgtggaggcctggcgggtgctttcaagcagaaactggtgcccttggtgcggacggtgtacgtccagaggccgaaacagaggaaccggcttccaggcaacttgttccagcaatggcgtgttcctctagaactccagatggcaagacaaatggctagctctggttcatcagggggcaaaatggataattctgtgttagtccttattgtgggcttatcaacaataggagctggtgcatatgcctacaaaactataaaagaagaccaaaaaagatacaatgaaagagtgatgggattaggactgtccccagaagagaaacagagaagagccattgcctccgctacagagggaggctcagttcctcagatcagggcaccaagtcacgtccctttcctgctgattggtggagggactgctgcttttgcagcagccagatccatccgggctcgggatcctggggccagggtcctgattgtatctgaagaccctgaactgccatacatgcgacctcctctttcaaaagaattgtggttttcagatgatccaaatgtcacaaagacactgcaattcagacagtggaatggaaaagagagaagcatatatttccagccaccttctttctatgtctctgctcaggacctgcctaatattgagaacggtggtgtggctgtcctcactgggaaaaaggtagtacatctggatgtaagaggcaacatggtgaaacttaatgatggctctcagattacctttgaaaagtgcttgattgcaacgggaggcactccaagaagtctgtctgccatcgatagggctggagcagaggtgaagagtagaacaacacttttcaggaagattggagattttagagccttggagaagatctctcgggaggtcaagtcaattacagttatcggcgggggcttccttgggagtgagctggcctgtgctcttggcagaaagtctcaagcctcgggcatagaagtgatccagctgttccctgagaaaggaaatatggggaagatccttcctcaatacctcagcaactggaccatggaaaaagtcaaacgagagggagtgaaagtgatgcccaatgcaattgtacaatcagttggagtcagcggtggcaggttactcattaagctgaaagatggaaggaaggtagaaactgaccacatagtgacagctgtgggcctagagcccaatgttgagttggccaagactggcggactggaaatagattccgattttggtggcttccgggtaaatgcagaactccaagcacgttctaacatctgggtggcaggggatgctgcatgcttctatgatataaagttgggtcgaaggcgagtagagcatcatgatcatgctgttgtgagtggaagactggctggagaaaacatgactggagccgctaagccatactggcatcagtcaatgttctggagtgatttgggtcctgatgtcggctatgaagctattggtctggtggatagtagtttgcccacagttggtgtttttgcaaaagcaactgcacaagacaacccaaaatctgccacagagcagtcaggaactggtatccgttcggagagtgagacagagtcagaagcttcggaaatcacaattcctcccagcgcccctgcagtcccacaggtccctgttgaaggggaggactacggcaaaggtgtcatcttctacctcagggacaaagttgtggtggggattgtgctatggaacgtctttaaccgaatgccaattgcaaggaagatcattaaggacggtgagcaacatgaagatctcaatgaagtagctaaactcttcaacattcatgaagattgaatcccaatcgtggaatacacaagcacttttccatccctggcg

SEQ ID NO: 2 - protein, 612 aa, mouse
MFRCGGLAGAFKQKLVPLVRTVYVQRPKQRNRLPGNLFQQWRVPLELQMARQMASSGSSGGKMDNSVLVLIVGLSTIGAGAYAYKTIKEDQKRYNERVMGLGLSPEEKQRRAIASATEGGSVPQIRAPSHVPFLLIGGGTAAFAAARSIRARDPGARVLIVSEDPELPYMRPPLSKELWFSDDPNVTKTLQFRQWNGKERSIYFQPPSFYVSAQDLPNIENGGVAVLTGKKVVHLDVRGNMVKLNDGSQITFEKCLIATGGTPRSLSAIDRAGAEVKSRTTLFRKIGDFRALEKISREVKSITVIGGGFLGSELACALGRKSQASGIEVIQLFPEKGNMGKILPQYLSNWTMEKVKREGVKVMPNAIVQSVGVSGGRLLIKLKDGRKVETDHIVTAVGLEPNVELAKTGGLEIDSDFGGFRVNAELQARSNIWVAGDAACFYDIKLGRRRVEHHDHAVVSGRLAGENMTGAAKPYWHQSMFWSDLGPDVGYEAIGLVDSSLPTVGVFAKATAQDNPKSATEQSGTGIRSESETESEASEITIPPSAPAVPQVPVEGEDYGKGVIFYLRDKVVVGIVLWNVFNRMPIARKIIKDGEQHEDLNEVAKLFNIHED

SEQ ID NO: 3 - protein, 11 aa, MOUSE
PPSAPAVPQVP

(19) United States
(12) Patent Application Publication
(10) Pub. No.: US 2004/0106119 A1
(43) Pub. Date: Jun. 3, 2004

(54) Crystal structure of the mouse apoptosis-inducing factor AIF and applications of such structural data to structure-based identification, screening, or design of AIF agonists or antagonists as well as AIF fragments, mutants or variants

(76) Inventor: Pedro Alzari, Paris (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/419,256

(22) Filed: Apr. 21, 2003

Related U.S. Application Data
(60) Provisional application No. 60/373,614, filed on Apr. 19, 2002.

(51) Int. Cl. C12Q 1/68; C07H 21/04; C12N 9/99; C12N 9/02

(52) U.S. Cl. 435/6; 435/184; 435/189; 536/23.2

(57) ABSTRACT

Structural features of Apoptosis-inducing Factor (AIF), a flavoprotein that can stimulate a caspase-independent cell-death pathway. Structure-based screening, identification and design of molecules that modulate AIF functional activities, including apoptosis and redox activity. Molecules useful for modulating apoptosis or AIF redox activity obtained by these methods.

### CROSS-REFERENCE TO RELATED APPLICATION

This application claims priority under 35 U.S.C. §120 to U.S. Provisional Application No. 60/373,614, filed Apr. 19, 2002.

### BACKGROUND OF THE INVENTION

1. Field of the Invention

Structural features of Apoptosis-inducing Factor (AIF), a flavoprotein that can stimulate a caspase-independent cell-death pathway. Structure-based screening, identification and design of molecules that modulate AIF functional activities, including apoptosis and redox activity. Molecules useful for modulating apoptosis or AIF redox activity obtained by these methods.

2. Description of the Related Art

Apoptosis-inducing factor (AIF) is a flavoprotein that is normally confined to mitochondria, but which can translocate to the cell nucleus where it induces apoptosis. Mitochondria play a key role in apoptosis by virtue of their capacity to release potentially lethal proteins. Another such latent death factor is cytochrome c, which can stimulate the proteolytic activation of caspase zymogens. Apoptosis-inducing factor (AIF) stimulates a caspase-independent cell-death pathway required for early embryonic morphogenesis. The nucleic acid and amino acid sequence of AIF is described by Susin et al., Nature 397: 441-446 (1999). AIF amino acid sequences are well conserved (e.g. >95%) between mice and humans.

Mature AIF is a flavoprotein of 57 kDa that shares significant homology with prokaryotic oxido-reductases, in particular NADH-dependent ferredoxin reductases from both bacteria and archaebacteria and also, with plant monodehydroascorbate reductases¹. In mammals, AIF is confined to mitochondria, the evolutionary relics of bacteria. Knock-out of the AIF gene disrupts the first wave of morphogenetic programmed cell death during early mouse embryo development, at the pluricellular stage, shortly after the differentiation of ectoderm and endoderm². An homolog of AIF has also been involved in differentiation-associated cell death of the facultatively multicellular slime mold Dictyostelium discoideum³. Thus, AIF must be considered as a phylogenetically ancient and ontogenetically early cell death regulator. Recent biochemical studies showed that both native and recombinant AIF exhibit NADH oxidase activity, leading to formation of the superoxide anion⁴. These data suggest that AIF belongs to the electron transferase class of flavoproteins⁵, its physiological role involving the transfer of electrons between so far unidentified redox partners. On the other hand, as other mitochondrial factors, AIF is released from mitochondria during apoptosis. AIF then migrates to the nucleus, thereby inducing chromatin condensation and large-scale DNA fragmentation⁹ by an unknown molecular mechanism. On isolated nuclei, this action appears to be independent from its oxido-reductase activity^{4,9}. Thus, as seen in cytochrome c, AIF may behave as a bifunctional protein with dissociable apoptogenic and redox properties. Yet, the nuclear events caused by AIF apparently depend on the apoptosis inducer² and the cell type^{10,11}, and are reversible at low apoptotic insult¹⁰.

To better understand the role of AIF in apoptosis and other cellular phenomena, screen biological response modifiers, or design molecules modulating AIF associated activities there is a need to determine the structure of AIF as well as to discover the effects of modifying the AIF structure.

### BRIEF DESCRIPTION OF THE INVENTION

To gain insight into the molecular modes of AIF function, one object of the invention is the identification of the structural features of AIF, including its secondary, tertiary and quaternary structure, especially those features that determine its functional activity.

Another object is the design of AIF mutants with altered functional activities based on AIF structural information. For instance, based on the structural information, key residues in a particular AIF domain can be altered to design AIF variants or mutants with altered functional activities. The invention also provides that such mutant or variant forms of AIF may be expressed in host cells or in transgenic animals.

Another object of the invention is the identification of key residues of AIF involved in its functional activity that may be used as targets for ligands, including drugs and antibodies, or as immunological determinants for the production of cellular or humoral response to AIF.

Another object of the invention is a method for the structured-based design of molecules, especially agonists or antagonists of AIF.

Other objects of the invention provide methods of screening molecules, including peptides or polypeptides that modulate AIF activities or methods of screening AIF mutants having altered functional activity.

### BRIEF DESCRIPTION OF THE DRAWINGS

FIGS. 1(a), (b) and (c) show the overall structure of murine AIF. FIG. 1(a) shows the various domains of AIF and compares AIF to BphA4 (a ferredoxin reductase component of biphenyl dioxygenase) and GR (glutathione reductase). FAD and NADH are shown in black and cyan sticks respectively. FIG. 1(b) shows the crystallographic contacts in AIF crystals. Monomers 2 and 3 form a crystallographic dimer related by a two-fold axis. The Proline-rich C-terminal insertion is stabilised by crystal contacts as seen between monomers 1 and 2. FIG. 1(c) shows the distribution of invariant residues (green) among mammalian and D. discoideum AIFs in both faces of the monomer. The FAD molecular surface is shown in magenta. The dimerization area is marked and an arrow depicts the two-fold axis.

FIGS. 2(a), (b), (c) and (d) refer to the structural details of AIF. FIG. 2(a) shows the FAD-binding site of AIF. The NADH molecule was position as observed in BphA4. Hydrogen bonds are shown in black and Van der Walls interactions, between E313 and FAD in red. FIG. 2(b) shows detail of the three salt bridges and the acid-acid pair that stabilise the N-terminal region of the C-terminal insertion in AIF and occlude Trp 482. FIG. 2(c) shows Trp fluorescence emission spectra of AIF, and two AIF point mutants: AIF-E313A and AIF-K176A. FIG. 2(d) shows the final 2mF₀-DFc electron density map around the FAD moiety and the two mutated residues (1.5-σ contour).

FIG. 3. The crystallographic AIF dimer. The stereo surface view of one crystallographic dimer is seen along the z-axis, slightly away from the non-crystallographic 2-fold axis. The rendered surface is colored by electrostatic potential. An asterisk (*) indicates the entrance of each NADH-binding pocket.

FIG. 4 shows the pattern of conserved residues at the molecular surface of AIF that could be involved in protein-ligand interactions. At the left, invariant aminoacids are shown in yellow for both faces of the monomer (top and bottom left). The C-terminal insertion characteristic of mammalian AIF is shown in violet. At the right, all basic amino acid residues (Arg, Lys) that are accessible for ligand-binding are shown in blue, superimposed onto the pattern of conserved (yellow) and non-conserved (red) residues.

FIG. 5 shows a close view of the structure of the NAD-binding pocket. The FAD moiety is seen at the bottom of the pocket (in light blue), and specific amino acid residues are labelled.

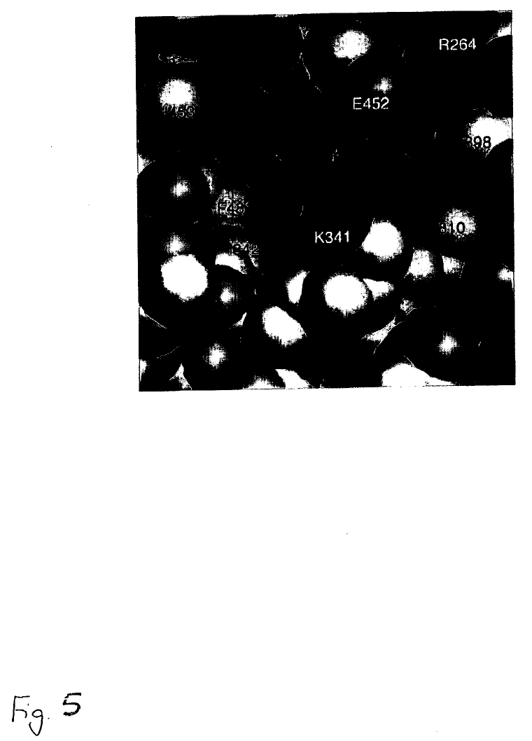

### DETAILED DESCRIPTION OF THE INVENTION

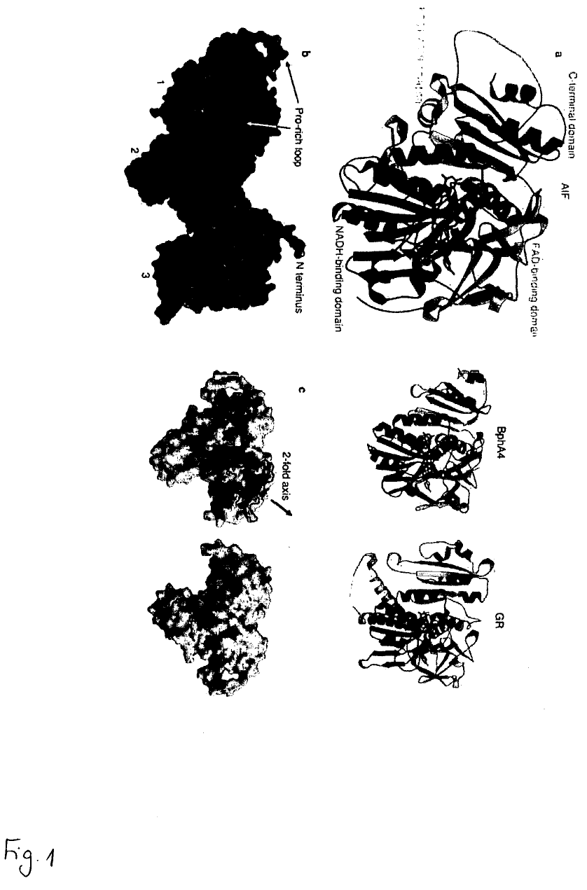

To gain insight into the molecular modes of AIF function, the crystal structure of AIF was determined using a truncated form of the mouse enzyme, AIFD1-121, lacking the N-terminal mitochondrial localisation sequence (Table 1, FIG. 1a). Mutagenesis and biochemical studies were carried out based on the structural information. Here, the crystal structure of mouse AIF at 2.0 Å is reported. Its active site structure and redox properties suggest that AIF functions as an electron transferase with a mechanism similar to that of bacterial ferredoxin reductases, its closest evolutionary homologs. However, it has been found that AIF structurally differs from these proteins in some essential features, including a long insertion in a C-terminal b-hairpin.

It has been found that the overall structure of AIF displays a glutathione reductase (GR)-like fold (FIG. 1a) and includes one FAD molecule per monomer. Similar to the enzymes of the GR-family, it has been determined that AIF is composed of three domains: an FAD-binding domain (residues 121-262 and 400-477), an NADH-binding domain (263-399), and a C-terminal domain (478-610), which in GR constitute most of its dimer interface. Both the FAD-binding and NADH-binding domains display the classical Rossmann fold, whereas the C-terminal domain is composed of five antiparallel b-strands (residues 477 to 579) followed by two a-helices (residues 580 to 610). Searches for structural similarity carried out with either the whole model or each separate domain show the closest match with BphA4, the ferredoxin reductase component of biphenyl dioxygenase from Pseudomonas sp. strain KKS102 (ref. 12). The root-mean-squares (rms) deviations for all equivalent Cα atoms of the two proteins is 2.5 Å, although the structural differences for the C-terminal domain (rmsd of 2.9 Å) are more important than those observed for the other two domains (rmsd ca 2 Å).

The structural comparison of the polypeptide backbones of AIF, BphA4 and E. coli GR emphasises their overall similarity (FIG. 1a). However, there are significant differences between the three proteins, the most remarkable being a long insertion in the C-terminal domain of AIF (residues 509-559) that is missing in the other two proteins. The N-terminal part of this insertion displays a defined secondary structure, namely two short helices that fold back onto the FAD-binding domain. It is followed by a long loop that adopts an open conformation, stabilised by crystal contacts with a neighbouring monomer (FIG. 1b). This C-terminal insertion seems to be a unique feature of mammalian AIF, since it is absent in all other proteins of related sequence, including the apoptosis-inducing factor recently identified in the mould Dyctiostelium discoideum³. This argues against a direct role of this insertion in the apoptogenic or redox properties of the protein. However, its open structure could indicate a putative binding site for chaperones such as Hsp70, which has been shown to interact with AIF, thereby precluding its apoptogenic effects ³. Moreover, the presence of a highly accessible proline-rich motif PPSAPAVPQVP (SEQ ID NO: 1) in this loop evokes the possibility of interactions with WW- or SH3-like domains, typically found in proteins liable to regulate a wide diversity of biological processes. A second region that bears significant differences among the three proteins is the one corresponding to two long a-helices in GR (residues 42 to 106), which are essential for catalysis and dimerization. These helices are missing in both AIF and BphA4, where the equivalent regions are shorter (47 residues in AIF, 25 in BphA4) and adopt a more extended conformation.

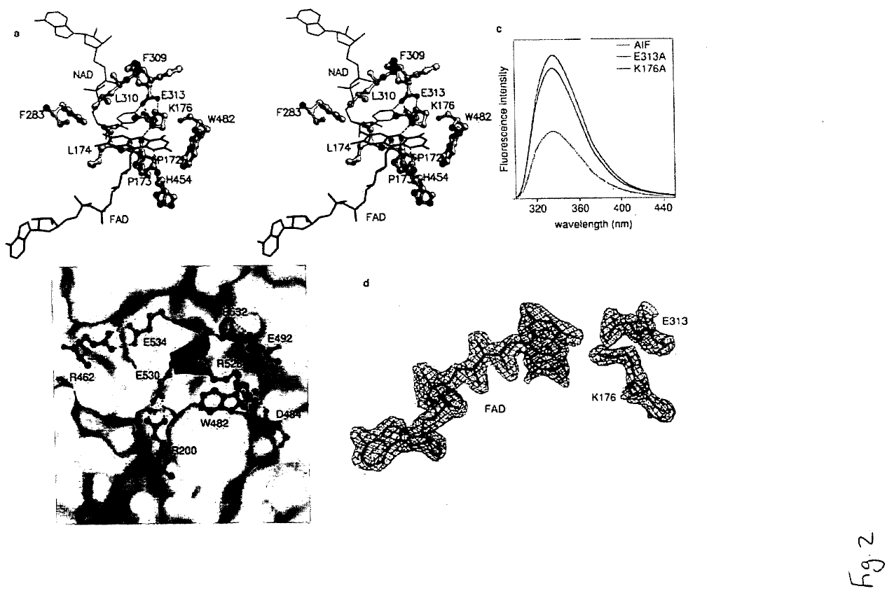

The FAD molecule binds non-covalently to AIF in an elongated manner (FIG. 2a), similar to that observed in BphA4 and slightly different from that in GR. The adenine nucleoside and the pyrophosphate group of the FAD are in contact with the most conserved region of the FAD-binding domain, whilst the isoalloxazine ring is partially accessible from the solvent in agreement with its role as a redox center. Its xylene moiety is located in a hydrophobic and well conserved solvent-shielded pocket, lined by residues Pro172, Pro173, Leu174, Phe283, Leu310 and the aliphatic portions of the side-chains of Arg171 and Arg284. In contrast, the environment of the pteridine moiety has a positive polar character that is thought to increase the flavin redox potential¹⁴.

As observed in other flavoproteins, the Ni and O2 positions of the isoalloxazine ring are within hydrogen bonding distance to one main-chain amide atom (His454) at the N-terminus of an a-helix, whose positive partial charge contributes to the stabilisation of the negative charge when the electrons are immersed in the flavin moiety¹⁵. Moreover, its N5 atom establishes a hydrogen bond with the N^{ζ} of Lys176 (3.0 Å), which in turn makes a salt bridge with Glu313 (2.8 Å), see FIGS. 2a and 2d. Interestingly, Lys176 displays slightly unfavourable values of the main-chain dihedral angles. This interaction pattern is conserved in BphA4 and GR-related enzymes, and has been proposed to play a functional role in hydride transfer¹⁵.

While the first step of the redox reaction (NADH oxidation) appears to be similar in the three reductases, the second step (FAD reoxidation) is clearly distinct. GR-like proteins have a conserved disulphide bridge that acts as an electron acceptor to oxidize the isoalloxazine ring. This additional redox centre is missing in both BphA4 and AIF. Instead, a stretch of three consecutive residues (Trp-Ser-Asp) on the si-side of the isoalloxazine ring is conserved among AIF and BphA4-like NADH-dependent reductases. The tryptophan residue in this motif (AIF Trp482) is largely exposed to the solvent in BphA4, and may be involved in the electron transfer route from FADH₂ to its physiological partner, ferredoxin ². In AIF, however, the helical region of the C-terminal insertion folds back onto the FAD-binding domain and completely occludes Trp482 from the bulk solvent (FIG. 2b). It could be argued that this conformation is stabilised by crystal packing forces and does not correspond to the native structure in solution. In particular, the carboxylate groups of two acidic residues, Glu532 and Glu492 are brought together within a priori unfavorable hydrogen-bonding distance of each other (2.4 Å) in the vicinity of Trp482 (FIG. 2b). Nevertheless, interactions between acidic side-chains are not rare in proteins and are sometimes found in active and binding sites¹⁶. Moreover, the steric and hydrophobic complementarity of the interacting surfaces between the C-terminal insertion and the protein core, as well as the formation of three additional salt bridges (Arg528-Asp484, Glu530-Arg200 and Glu534-Arg462, see FIG. 2b), strongly suggest that the conformation found in the crystal is maintained in solution. In fact, the characteristics of tryptophan fluorescence emission of AIF confirm that all the tryptophan residues in the molecule are buried (FIG. 2c), according to Burstein's classification¹⁷. Also, the absence of a iodide dynamic quenching effect on the tryptophan fluorescence emission of AIF supports the idea that, in solution, the protein does not contain any fully exposed tryptophan residues.

A comparison of the NAD-binding domains of GR, BphA4 and AIF reveals that the nicotinamide binding site is more conserved than that of the adenine moiety. For instance, the gate-and-anchor role played by Tyr177 in GR may be fulfilled, in AIF, by Phe309. Yet, a representation of the surface charges in the NAD-pocket clearly shows some important differences between AIF and BphA4. The most striking difference is the absence of a residue equivalent to BphA4 Arg183, which makes hydrogen bonds with both the pyrophosphate group and the ribose moiety of NADH. Interestingly, this arginine residue defines one of the walls of the NADH-binding pocket in BphA4. As a consequence, AIF possesses a comparatively larger pocket with fewer specific contacts for NADH. This suggests a weaker NADH binding consistent with the difficulty to model the ligand in the electron density maps. The presence of a bigger pocket may also indicate a binding site for an unknown substrate that could be reduced by the FADH₂.

To gain further insight into the redox properties of AIF, two point mutants, AIF-E313A and AIF-K176A were produced. These mutants were expected to have an effect on hydride transfer and FAD fixation. Indeed, when they are prepared under the conditions used for AIFD1-101, they tend to loose the flavin cofactor, and yield the corresponding apo-proteins. However, when FAD was added to the purification buffers, active holo-proteins were obtained. The redox kinetic parameters of these mutants and AIFD1-101 are summarised in Table 2. Both point mutants show a higher k_{cat} than the wild-type, but the most striking feature was exhibited by AIF-E313A, whose apparent Kₘ for NADH falls by a factor of 20, thus resulting in a net gain (30-fold) of catalytic efficiency. This improvement could arise from structural rearrangements in the active site facilitating a direct hydride transfer between the C4a position of NADH and the N5 of FAD¹⁸. Tryptophan fluorescence emission experiments show that in both point mutants the flavin less effectively quenches the tryptophan emission (FIG. 2c). This is probably due to an increased mobility of the isoalloxazine ring that would reduce the Forster's energy transfer from tryptophan residues to the flavin. These results are in agreement with the loss of the prosthetic group during the purification of the mutant proteins.

Little attention has been given to the physiological role of AIF within the mitochondria. To gain some insight, it may be enlightening to consider the partners of BphA4, which is closely related to AIF both phylogenetically and structurally. BphA4 reduces the ferredoxin component (BphA3) of the biphenyl dioxygenase complex. BphA3 is a Rieske protein similar to other iron-sulfur proteins (ISP) found in mitochondria, such as the ISP from the cytochrome bc1 complex. Indeed, the globular domain of this ISP is exposed to the mitochondrial intermembrane space and could therefore be accessible to AIF. Moreover, the overall structure of ISP from bovine cytochrome bc1 BphF, Iwata, S., Science 281:64-71 (1998), is remarkably similar to that of Burkholderia sp. BphF, Colbert, C. I., Structure Fold Des. 8(12): 1267-78 (2000), a homolog of BphA4 (72% sequence identity). Although it is well known that the cytochrome bc1 complex catalyzes the electron transfer from ubihydroquinone to cytochrome c in the mitochondrial respiratory chain, it is tempting to speculate on a possible role of AIF in this essential cellular process.

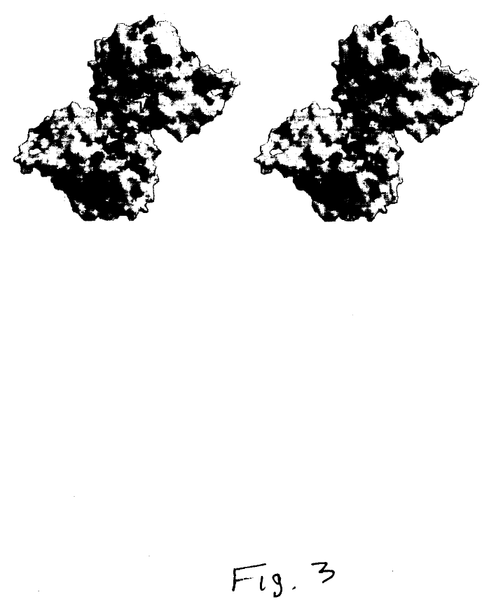

The molecular mode of action of AIF in apoptosis is open to much speculation^{2,9,10,13,19}. In the nucleus, AIF could be directly responsible for large-scale chromatin fragmentation through free radical-mediated DNA cleavage, or indirectly through the recruitment and activation of other factor(s) conveying a nuclease activity. In principle, the first hypothesis appears less likely, since previous results suggest that the apoptogenic and redox AIF activities might be dissociable from each other^{4,9}. It may be hypothesised that the apoptogenic role of AIF could involve protein-DNA interactions. Yet, there are no obvious DNA-binding structural motifs in mouse AIF. It has been proposed that the C-terminus of D. discoideum AIF could include a helix-turn-helix motif 3. However, the spatial arrangement of the corresponding helices in mouse AIF is different from that found in known DNA-binding domains of this type, and the C-terminal insertion could interfere with putative intermolecular interactions involving these helices. Furthermore, the surface distribution of basic (Arg/Lys) residues that are conserved in different AIF sequences or the electrostatic potential at the molecular surface (FIG. 3) did not reveal a particular pattern suggestive of a DNA-binding site.

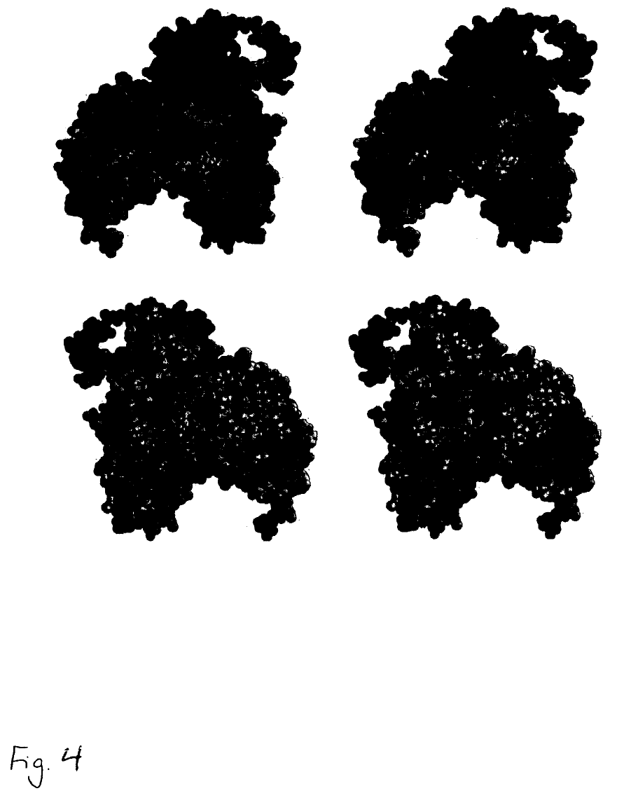

The possibility that AIF could recruit other protein factors—such as nucleases—involved in apoptosis has also been invoked. The comparative sequence analysis of mammalian and D. discoideum AIFs revealed that the invariant residues tend to be clustered in patches at the molecular surface, suggesting putative binding targets for those putative apoptotic factors (FIG. 4). Interestingly, these conserved patches are concentrated on the face of the molecule that includes the neighbourhood of the NADH-binding pocket and the interface of the crystallographic dimer (FIG. 1c). As shown in FIG. 3, this dimer has an overall saddle shape with the conserved residues lining its concave surface. Although recombinant AIF behaves as a monomer in solution (as determined by analytical ultracentrifugation in the range of micromolar protein concentration), it is tempting to speculate that AIF could dimerize upon interaction with a putative partner, protein or DNA, or after post-translational modification. Indeed, an essentially identical dimer was observed in BphA4 crystals¹², a known dimeric protein. Whatever the case, these invariant surface areas may indicate target sites for other factors, possibly involved in the AIF-mediated apoptosis pathway.

The identification of AIF structures described herein, as well as the differences found with non-apoptogenic homologs, permit the identification of the exact metabolic and cytocidal functions acquired by AIF during evolution and the engineering of molecules that modulate various AIF activities or the design of AIF variants with altered functional activity or the identification of AIF fragments of interest. In a preferred embodiment such a fragment comprises at least 4 amino acids.

Methods for producing and screening AIF mutants or variants are well known in the art and are also described by Current Protocols in Molecular Biology (1987-2002), vols. 1-4. Such mutants or variants may contain point mutations of 1 or more amino acid residues of the AIF amino acid sequence, including deletions, insertions or substitutions of a particular residue. The AIF sequence, may advantageously be altered at only a few residues, e.g. at 1, 2, 3, 4 or 5 residues, up to 20-50 residues or more or so long as the variant retains a desired structural feature or desired functional activity. Fragments of AIF, including peptides modified to improve their biological stability, are also contemplated.

Nucleic acids encoding AIF or AIF variants may also be configured to contain regulatory sequences, such as ribosome binding sites, promoters or other regulatory sequences useful for modulating their expression, for instance, for up-regulating AIF expression. Such regulatory sequences are also well-known in the art and are described by Methods for producing and screening AIF mutants or variants are well-known in the art and are also described by Current Protocols in Molecular Biology (1987-2002), vols. 1-4. Generally, a nucleic acid sequence encoding an AIF variant will have 70%, preferably 80%, more preferably 90, 95 or 99% similarity to a native AIF sequence as determined by. Such similarity may be determined by an algorithm, such as those described by Current Protocols in Molecular Biology, vol. 4, chapter 19 (1987-2002) or by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2: 482-489 (1981), to find the best segment of identity or similarity between two sequences. Gap performs global alignments: all of one sequence with all of another similar sequence using the method of Needleman and Wunsch, J. Mol. Biol. 48:443-453 (1970). When using a sequence alignment program such as BestFit, to determine the degree of sequence homology, similarity or identity, the default setting may be used, or an appropriate scoring matrix may be selected to optimize identity, similarity or homology scores. Alternatively, sequence alignment of AIF variants can be produced from 3D models of the protein, using the structural information reported here, in order to optimise the alignment of specific features of the structure such as critical functional residues or secondary structure elements. Software for the purpose is available, for instance, as QUANTA or INSIGHT from Molecular Structure Inc.

Such AIF variants may also be characterised in that a nucleic acid sequence encoding such a variant will hybridize under stringent conditions with a native AIF sequence, such as the native human or murine AIF sequence. Such hybridization conditions may comprise hybridization at 5×SSC at a temperature of about 50 to 68° C. Washing may be performed using 2×SSC and optionally followed by washing using 0.5×SSC. For even higher stringency, the hybridization temperature may be raised to 68° C. or washing may be performed in a salt solution of 0.1×SSC. Other conventional hybridization procedures and conditions may also be used as described by Current Protocols in Molecular Biology, (1987-2002), see e.g. Chapter 2. Such variants may be expressed in a suitable host cell and such host cells used to screen drugs or other compounds for an ability to modulate AIF activity.

Methods for making transgenic or animals with knock-out mutations are well known in the art and may be used to produce animals expressing variant forms of AIF. Reference is also made to Current Protocols in Molecular Biology (1987-2002), vols 1-4, especially vol. 4, chapter 23. Such animals may be used to further elaborate on AIF functions, or to screen drugs or other compounds for the ability to modulate AIF functional activity.

Epitopes and other immunological determinants of AIF may be identified by conventional means. Such determinants may be used to induce humoral or cellular immune responses to AIF or fragments of AIF and modulate the functional activity of AIF.

Methods for assaying the redox and apoptotic activities of AIF and AIF variants are well-established in the literature. The redox activity can be assessed using available protocols to measure NAD(P)H oxidase activity, NBT reduction activity, and/or the production of free radicals, as described by reference 4 below. The apoptotic activity of AIF variants can be assessed using a cell-free system, in which purified HeLa cell nuclei are exposed to the protein and the ensuing nuclear apoptosis is then quantified by cytofluorometric determination of DNA content and/or pulsed-field gel electrophoresis, as described for example by Susin et al., J. Exp. Med. 186: 25-37 (1997). Other methods can also be used to measure the apoptotic activity, such as microinjection of the protein in cultured cells or immunofluorescence analysis using specific monoclonal antibodies, as described for example by Susin et al., J. Exp. Med. 189:381-393 (1999). These redox and apoptotic assays may be used to further assess the functional role of specific AIF mutants based on 3D structure, or to screen for chemical compounds that are able to impair or abolish one or more AIF functional activities. Once suitable compounds are identified, their functional properties can be further optimized by structure-based drug design methods using the structural information reported here.

### EXAMPLES

Protein Expression and Purification.

The deletion mutant AIFD1-121, which corresponds to the mature protein and retains both apoptotic and redox activities⁴, was produced as described⁹. To investigate the redox activity of AIF, a recombinant protein was constructed by subcloning the DNA coding for the mature murine protein (residues 102-610) in the pET28a (NOVAGEN) expression vector, providing an N-terminal His-tag. The AIF-E313A and AIF-K176A mutants were obtained from that base construct by site-directed mutagenesis. All these proteins were overexpressed in E. coli BL21(DE3), and purified on Ni-IMAC columns, in the presence of 100 μM FAD.

Crystallisation and Structure Determination.

AIFD1-121 was crystallised in hanging drops containing 18% PEG-5000, 80 mM MgCl₂, 50 mM HEPES, pH 7.75, both in the presence and absence of NAD(P)⁺. Yellow plate-like crystals belonging to the orthorhombic P2₁2₁2₁ or monoclinic P2₁ space groups, and containing two monomers per asymmetric unit in each case, were obtained.

Diffraction data sets were collected using synchrotron radiation at ESRF (Grenoble, France) beamline ID14.4 for the AIF-NADP⁺ complex in the orthorhombic (a=86.3 Å, b=109.9 Å, c=114.6 Å) and monoclinic (a=64.5 Å, b=86.3 Å, c=99.7 Å, b=98.9°) space groups. A Multiwavelength Anomalous Diffraction (MAD) data set was also collected at the same beamline for a mercurial derivative of the orthorhombic form (a=87.1 Å, b=112.5 Å, c=112.7 Å) at three different wavelengths. All data sets were integrated and reduced using the programs MOSFLM²⁰ and SCALA²¹.

The 3D structure was solved by a combination of MAD and Molecular Replacement (MR) techniques. Four heavy atom sites were found using Patterson methods with the program SHELXS22 and refined with MLPHARE²¹ (FOMcen=0.56 and FOMacen=0.50). However, the resulting map after density modification with the program DM²¹ was too discontinuous to allow polypeptide chain tracing. In parallel, a poorly contrasted MR solution (correlation factor of 0.18) was found for the monoclinic space group using the program AMoRe²³ and the structure of BphA4 from Pseudomonas sp. strain KKS102 (PDB entry:1D7Y, 21% of amino acid identity with AIF) as a search probe. The electron density map corresponding to a single monomer in the monoclinic space group was subsequently used as a search probe in MR calculations to solve the structure of the mercurial derivative in the orthorhombic space group. Difference Fourier maps calculated with MR phases at this stage clearly revealed the four independent heavy atom binding sites respectively close to four of the six cysteine residues present in the crystallographic dimer, thus confirming the correctness of the MR solution.

The poor quality of the MR model (Rfactor>50%) prevented direct atomic refinement, but an electron density map calculated with combined (model-MAD) phases allowed to retrace the polypeptide chain for 60% of the model. Iterative model refinement and rebuilding were subsequently carried out using the programs REFMAC²⁴ and XtalView²⁵ for the inspection of combined-phases and (2mFo-DFc) maps. Crystallographic refinement was independently performed for the three data sets (Table 1). The final model includes amino acid residues 122-610, with most main- and side-chains unambiguously defined in the electron density. The backbone dihedral angles of all but one non-glycine residues in each monomer fall in the more favorable or additionally allowed regions of the Ramachandran plot, with the only exception of Thr533 in the C-terminal insertion. This threonine residue is well defined in density and is constrained by the strong interactions done by the two adjacent residues, Glu532 and Glu534 (FIG. 2b). No ions were found in the structure. Although it was not possible to model either the NAD or NADP-bound molecules, some extra density in their expected pocket suggests an incomplete occupancy of these ligands.

Since no significant differences were found between the three final models, all the analysis was performed with that derived from the orthorhombic native dataset. Structural similarity searches were performed with the DALI server (http://www.ebi.ac.uk/dali). Electrostatic calculations were done with DELPHI²⁶. Figures were drawn using XtalView²⁵, GRASP²⁷, Molscript and Raster3D 946-50 (1991).

Redox activity and Fluorescence Assays.

The kinetic parameters for the redox activity were determined by varying NADH concentration (from 5 μM to 2.5 mM) in the presence of an excess of 2,2′-di-p-nitrophenyl-5-5′-diphenyl-3,3′[3-3′-dimetoxy-4-4′difenilen]tetrazolium chloride (NBT), in 0.1 M Tris buffer, pH 8.0. Optical absorbance measurements at 540 nm were performed on a Hewlett Packard 8452 A UV-visible spectrophotometer, and an extintion coefficient of 7.2 mM⁻¹cm⁻¹ was used for formazan blue at this wavelength. Corrected steady-state Trp fluorescence emission spectra were recorded on a SLM Aminco Series 2 spectrophotometer. The excitation and emission spectral bandwidths were 4 nm. In order to reduce the tyrosine contribution to the Trp fluorescence emission, the excitation wavelength used was 295 nm. The fluorescence was observed through a Schott cut-off filter WG 320, and the Raman light scattering from the buffer was substracted from the fluorescence spectra of each sample.

Structural data deposition. Atomic co-ordinates and structure factor amplitudes have been deposited in the Protein Data Bank under accession number 1GV4. As used herein, the terms “structure-based” or “structure-based design” refers to molecules derived, for instance, from data deposited under this accession number.

Modifications and Other Embodiments

Various modifications and variations of the described AIF products and the described methods, as well as the concept of the invention will be apparent to those skilled in the art without departing from the scope and spirit of the invention. Although the invention has been described in connection with specific preferred embodiments, it should be understood that the invention as embodied is not intended to be limited to such specific embodiments. Various modifications of the described modes for carrying out the invention which are obvious to those skilled in the immunological, molecular biological, medical, biological, chemical or pharmacological arts or related fields are intended to be within the scope of the following embodiments.

Incorporation by Reference

Each document cited by or referred to in this disclosure is incorporated by reference in its entirety. Specifically, the contents of U.S. Provisional Application No. 60/373,614 are hereby incorporated by reference.

TABLE 1
Final refinement and model statistics
Data collection

| Data set | MAD λ1 | MAD λ2 | MAD λ3 | native | native |
|---|---|---|---|---|---|
| Space group | | P2₁2₁2₁ | | P2₁2₁2₁ | P2₁ |
| Wavelength (Å) | 1.0068 | 1.0088 | 0.9465 | 0.933 | 0.933 |
| Resolution (Å) | 20-2.2 Å | 20-2.2 Å | 20-2.0 Å | 20-2.0 Å | 20-2.2 Å |
| Completeness (%)¹ | 97.2(86.3) | 97.8(89.3) | 98.7(85.3) | 96.5(92.6) | 98.2(96.6) |
| Rₘₑₐₛ¹ | 0.116(0.352) | 0.092(0.201) | 0.111(0.341) | 0.110(0.273) | 0.106(0.288) |

Refinement

| | | | |
|---|---|---|---|
| Unique reflections | 75291 | 71317 | 53783 |
| R_{factor}^{1,2} | 19.8 (23.6) | 21.6 (24.0) | 20.1 (22.2) |
| R_{free}^{1,2} | 24.2 (30.6) | 25.7 (33.9) | 24.7 (31.0) |
| N. of protein atoms | 7463 | 7442 | 7406 |
| N. of solvent atoms | 448 | 306 | 361 |
| R.m.s deviations | | | |
| Bonds (Å) | 0.02 | 0.02 | 0.02 |
| Angles (°) | 1.97 | 1.77 | 1.98 |

¹Numbers in parentheses correspond to the highest resolution shell
²R_{factor} = Sₕₖ₁∥F_{obs}|−k|F_{calc}∥/Sₕₖ₁|F_{obs}|; free R_{factor}, same for a test set of 5% reflections not used during refinement.

TABLE 2
NET reductase activity steady-state parameters

| | | | | | | k_{cat}/ |
|---|---|---|---|---|---|---|
| | Concen- | Vₘₐₓ | | | | Kₘ |
| | tration | (nM | Kₘ | | k_{cat} | (mM⁻¹ |
| AIF | (nM) | min⁻¹) | (μM) | U/mg | (min⁻¹) | min⁻¹) |
| AIF | 503 | 13 | 173 | 0.90 | 52 | 300 |
| Δ1-101 | | | | | | |
| E313A | 736 | 26 | 8 | 1.22 | 70 | 8900 |
| K176A | 494 | 18 | 140 | 1.20 | 72 | 530 |

### REFERENCES

1. Lorenzo, H. K., Susin, S. A., Penninger, J. & Kroemer, G. Cell Death Differ. 6, 516-24 (1999).

2. Joza, N. et al. Nature 410, 549-54 (2001).

3. Amoult, D. et al. Mol. Biol. Cell 12, 3016-30 (2001).

4. Miramar, M. D. et al. J. Biol. Chem. 276, 16391-16398 (2001).

5. Massey, V. J. Biol. Chem. 269, 22459-62 (1994).

6. Bernardi, P., Petronilli, V., Di Lisa, F. & Forte, M. Trends Biochem. Sci. 26, 112-7 (2001).

7. Loeffler, M. & Kroemer, G. Exp. Cell Res. 256, 19-26 (2000).

8. Green, D. R. & Reed, J. C. Science 281, 1309-12 (1998).

9. Susin, S. A. et al. Nature 397, 441-6 (1999).

10. Dumont, C. et al. Blood 96, 1030-8 (2000).

11. Zhou, G. & Roizman, B. J. Virol. 74, 9048-53 (2000).

12. Senda, T. et al. J. Mol. Biol. 304, 397-410 (2000).

13. Ravagnan, L. et al. Nat. Cell Biol. 3, 839-43 (2001).

14. Ghisla, S. & Massey, V. Eur. J. Biochem. 181, 1-17 (1989).

15. Pai, E. F. & Schulz, G. E. J. Biol. Chem. 258, 1752-7 (1983).

16. Flocco, M. M. & Mowbray, S. L. J. Mol. Biol. 254, 96-105 (1995).

17. Reshetnyak, Y. K. & Burstein, E. A. Biophys. J 81, 1710-34 (2001).

18. Fraaije, M. W. & Mattevi, A. Trends Biochem. Sci. 25, 126-32 (2000).

19. Susin, S. A. et al. G. J. Exp. Med. 192, 571-80 (2000).

20. Leslie, A. G. W. Joint CCP4 and ESF-EACBM Newsletters on Protein Crystallography 26 (1992).

21. Collaborative Computational Project Number 4. Acta Crystallogr. D 50, 760-3 (1994).

22. Sheldrick, G. M. Methods Enzymol. 276, 628-41 (1997).

23. Navaza, J. Acta Crystallogr. D 50, 157-63 (1994).

24. Murshudov, G. N., Vagin, A. A. & Dodson, E. J. Acta Crystallogr. D 53, 240-55 (1997).

25. McRee, D. E. J. Mol. Graph. Model 10, 44-6 (1992).

26. Sharp, K. A. & Honig, B. Annu. Rev. Biophys. Biophys. Chem. 19, 301-32 (1990).

27. Nicholls, A., Sharp, K. A. & Honig, B. Proteins 11, 281-96 (1991).

28. Kraulis, P. J. J. Appl. Cryst. 24, 946-50 (1991).

29. Merritt, E. A. & Bacon, D. J. Methods Enzymol. 277, 505-24 (1997).

30. Earnshaw, W. C., Nature 397, 387-389 (1999).

31. Iwata, S., Science 281:64-71 (1998).

32. Colbert, C. I., Structure Fold Des. 8(12): 1267-78 (2000).

What is claimed is:

1 A polypeptide that modulates apoptosis or AIF redox activity comprising a variant of AIF or a fragment thereof.

2 The polypeptide according to claim 1, wherein it is at least partially identified, screened, designed or engineered using the data deposited in the Protein Data Bank under accession number 1GV4.

3 The polypeptide according to claim 1, wherein it has at least 70% homology with native AIF.

4 The polypeptide according to claim 1, wherein it has at least 80% homology with native AIF.

5 The polypeptide according to claim 1, wherein it has at least 90% homology with native AIF

6 The polypeptide according to claim 1, wherein it is encoded by a nucleic acid that hybridizes under stringent conditions with a nucleic acid encoding native AIF.

7 The polypeptide according to claim 1, wherein it comprises at least one variant of the FAD binding domain, residues 122-262, of AIF.

8 The polypeptide according to claim 1, wherein it comprises at least one variation of the NAHD binding domain, residues 478-610, of AIF.

9 The polypeptide according to claim 1, wherein it comprises at least one variation of the C-terminal domain, residues 478-610, of AIF.

10 A polypeptide comprising at least one variation of residues 509-559 of AIF.

11 The polypeptide according to claim 1 wherein its sequence is SEQ ID No.: 1.

12 The polypeptide according to claim 1, wherein it comprises a variation that decreases turn-over of AIF.

13 The polypeptide according to claim 1, wherein it comprises a variation that increases turn-over of AIF.

14 The polypeptide according to claim 1, wherein it has decreased interaction with another protein compared to AIF.

15 The polypeptide according to claim 14, wherein it comprises at least one variation of residues 509-559 of AIF

16 The polypeptide according to claim 1, wherein it has a decreased ability to bind to a chaperonin or a heat shock protein compared to AIF.

17 The polypeptide according to claim 1, wherein it has a decreased ability to bind a protein containing an SH3 or WW module.

18 The polypeptide according to claim 1, wherein it comprises at least one variation or modification of SEQ ID No.: 1.

19 The polypeptide according to claim 1, wherein it has increased interaction with other proteins compared to AIF.

20 The polypeptide according to claim 1, wherein, it has an increased ability to bind to a chaperonin or a heat shock protein compared to AIF.

21 The polypeptide according to claim 20, wherein it comprises at least one variation or modification of residues 509 to 559 of AIF.

22 The polypeptide according to claim 1, wherein it has increased ability to bind to a protein containing an SH3 or WW module compared to AIF.

23 The polypeptide according to claim 22, wherein it comprises at least one modification of SEQ ID NO.: 1.

24 The polypeptide according to claim 1, wherein it is less efficiently transported into the nucleus of a cell than native AIF.

25 The polypeptide according to claim 1, wherein it is more efficiently transported into the nucleus of a cell than native AIF.

26 The polypeptide according to claim 1, wherein it modulates AIF redox activity.

27 The polypeptide according to claim 1, wherein it has increased AIF redox activity compared with native AIF.

28 The polypeptide according to claim 1, wherein it has increased AIF redox activity compared with native AIF.

29 The polypeptide according to claim 26, wherein it comprises at least one modification of residues 263-399 of AIF.

30 The polypeptide according to claim 26, wherein it comprises residues 263 to 399 having at least one mutation at residue 319.

31 The polypeptide according to claim 1, wherein it comprises at least one epitope of AIF or at least one T-cell determinant of AIF.

32 A nucleic acid encoding the polypeptide according to claim 1.

33 A method for identifying a compound that modulates apoptosis or AIF redox activity or which is an AIF agonist or antagonist comprising (A) contacting said compound with AIF or a fragment or variant thereof and measuring the interaction of said compound with AIF or a fragment or variant thereof; (B) contacting said compound with a cell expressing AIF or a fragment or variant thereof and measuring the interaction of said compound with said cell expressing AIF or a fragment or variant thereof; or (C) comprising contacting said compound with an animal expressing AIF or a fragment or variant thereof and measuring the interaction of said compound with AIF; or (D) identifying a compound having a three dimensional structure similar to AIF or to a domain of AIF consistent with the data deposited in the Protein Data Bank under accession umber 1GV4.

34 The method of claim 33 comprising (A) contacting said compound with AIF or a fragment or variant thereof and measuring the interaction of said compound with AIF or a fragment or variant thereof.

35 The method of claim 34 comprising contacting said compound with AIF or a fragment thereof and measuring the interaction of said compound with an AIF site identified as a site of interest using the data deposited in the Protein Data Bank under accession number 1GV4.

36 The method of claim 34 comprising contacting said compound with the C-terminal domain of AIF or a fragment or variant thereof.

37 The method of claim 33 comprising (B) contacting said compound with a cell expressing AIF or a fragment or variant thereof and measuring the interaction of said compound with AIF or a fragment or variant thereof.

38 The method of claim 37 comprising contacting said compound with a cell expressing AIF or a fragment thereof and measuring the interaction of said compound with an AIF site identified as a site of interest using the data deposited in the Protein Data Bank under accession umber 1GV4.

39 The method of claim 37 comprising contacting said compound with a cell expressing the C-terminal domain of AIF or a fragment or variant thereof.

40 The method of claim 33 comprising (C) contacting said compound with an animal expressing AIF or a fragment or variant thereof and measuring the interaction of said compound with AIF.

41 The method of claim 40 comprising contacting said compound with an animal expressing AIF or a fragment thereof and measuring the interaction of said compound with an AIF site identified as a site of interest using the data deposited in the Protein Data Bank under accession umber 1GV4.

42 The method of claim 40 comprising contacting said compound with an animal expressing the C-terminal domain of AIF or a fragment or variant thereof.

43 The method of claim 33 for identifying an AIF agonist or antagonist comprising (D) identifying a compound having a three dimensional structure similar to AIF or to a domain of AIF consistent with the data deposited in the Protein Data Bank under accession umber 1GV4.

44 The method of claim 43 comprising identifying a compound having a three dimensional structure similar to the C-terminal domain of AIF and testing said compound for AIF agonistic or antagonistic activity.

45 A compound identified by the method of claim 33.

46 A method for the preparation of a compound that modulates apoptosis or AIR redox activity, comprising the following steps:
a) identifying a compound by a method according to claim 33, and
b) synthesizing the compound identified in step (a).

47 A method for modulating apoptosis in a mammal comprising administering the compound of claim 45 to said mammal.

48 The method according to claim 47, wherein said compound induces increased apoptosis.

49 The method according to claim 47, wherein said compound induces decreased apoptosis.

50 The method according to claim 47, wherein said mammal is human.

51 A method for modulating redox activity in a mammal comprising administering the compound of claim 45 to a mammal.

52 The method according to claim 51, wherein said compound induces increased redox activity.

53 The method according to claim 51, wherein said compound induces decreased redox activity.

54 The method according to claim 51, wherein said mammal is human.

55 A method for the design of a molecule having AIF agonist or antagonist activity, wherein said method comprises the use of the data deposited in the Protein Data Bank under accession number 1GV4.

56 A molecule obtained by the method of claim 55.

57 A method for the preparation of a compound having AIF agonist or antagonist activity comprising the following steps:
a) designing a compound by the method according to claim 55, and
b) synthesizing the compound designed in step (a).

58 A method for the identification of a fragment or a variant of AIF of interest, wherein said method comprises the use of the data deposited in the Protein Data Bank under accession number 1GV4.

59 Software comprising the use the data deposited in the Protein Data Bank under accession number 1GV4 to predict, design or engineer AIF sites of interest.

60 A computer-readable medium encoded with a first set of a plurality of computer readable values that correspond with the data deposited in the Protein Data Bank under accession number 1GV4, wherein said plurality of computer readable values are arranged such that when retrieved by a processor, said processor is configured to present a visual display signal that when input into a display presents a visual representation of a protein or polypeptide structure.

61 A computer-readable medium encoded with a first set of a plurality of computer readable values that correspond with the data deposited in the Protein Data Bank under accession number 1GV4, wherein said plurality of computer readable values are arranged such that when retrieved by a processor, said processor is configured to compare said values with a second set of computer readable values representing a compound, and determine the degree of correspondence between said first set of values and said second set of values, wherein the degree of similarity of said first and second set of values correlates with the degrees of similarity of said compound with AIF.

62 A computerized method for selecting or identifying a compound with AIF agonist or antagonist activity comprising comparing data representing at least one structural feature of AIF deposited in the Protein Data Bank under accession number 1GV4 with data representing the molecular structure of one or more compounds to be evaluated, and selecting a compound having a molecular structure similar within a set of predetermined parameters to at least one structural feature of AIF.

63 The method of claim 62, wherein the structural feature is a secondary molecular structure.

64 The method of claim 62, wherein the structural feature is a tertiary molecular structure.

65 The method of claim 62, wherein the structural feature is a quaternary molecular structure.

66 A computerized method for selecting or identifying an AIF fragment or variant comprising comparing data representing at least one structural feature of AIF deposited in the Protein Data Bank under accession number 1GV4 with data representing the molecular structure of at least one variant or fragment thereof of AIF to be evaluated and selecting a variant or fragment thereof based on similarity or divergence of the structure of said compound with the structure of AIF.

67 The method of claim 66, wherein the structural feature is a secondary molecular structure.

68 The method of claim 66, wherein the structural feature is a tertiary molecular structure.

69 The method of claim 66, wherein the structural feature is a quaternary molecular structure.
